(19) United States
(12) Patent Application Publication
(10) Pub. No.: US 2005/0033163 A1
(43) Pub. Date: Feb. 10, 2005

(54) Intrauterine implant and methods of use

(75) Inventors: Douglas J. Duchon, Chanhassen, MN (US); Paul Danielson, Shakopee, MN (US); James Presthus, Edina, MN (US)

Correspondence Address: INSKEEP INTELLECTUAL PROPERTY GROUP, INC, 1225 W. 190TH STREET, SUITE 205, GARDENA, CA, 90248, US

(73) Assignee: ImPres Medical, Inc.

(21) Appl. No.: 10/850,761

(22) Filed: May 21, 2004

Related U.S. Application Data
(63) Continuation-in-part of application No. 10/726,433, filed on Dec. 3, 2003.
Application No. 10/726,433 is a continuation of application No. 09/840,951, filed on Apr. 24, 2001, now Pat. No. 6,708,056.
(60) Provisional application No. 60/472,644, filed on May 21, 2003.

(51) Int. Cl. A61B 6/00

(52) U.S. Cl. 600/431

(57) ABSTRACT

Devices and methods of use or treatment are disclosed for creating fibrosis and resulting in amenorrhea. In particular, the device relates to an easily deployed intrauterine implant that readily and consistently reduces or eliminates abnormal intrauterine bleeding. In addition, the device is also used as a uterine marker for visualizing endometrial tissue thickness and potential changes. The methods of the present invention serve as a supplement to or a replacement for conventional treatments and procedures used to treat menorrhagia.

This application claims priority to U.S. Provisional Application Ser. No. 60/472,644 filed May 21, 2003 entitled Intrauterine Implant and Methods of Use. This application is a continuation-in-part of U.S. application Ser. No. 10/726,433 filed Dec. 3, 2003 entitled Method and Apparatus for Creating Intrauterine Adhesions which is a continuation of U.S. application Ser. No. 09/840,951 filed Apr. 24, 2001 entitled Method and Apparatus for Creating Intrauterine Adhesions. All of the above applications are incorporated herein by reference.

### BACKGROUND OF THE INVENTION

Nearly all women, at some time during their reproductive life, experience some type of menstrual disorder. These disorders range from mild to severe, often resulting in numerous lost work hours and the disruption of personal/family life each month. In general, physical symptoms such as bloating, breast tenderness, severe cramping (dysmenorrhea) and slight, temporary weight gain frequently occur during most menstrual cycles. In addition to physical symptoms, emotional hypersensitivity is also very common. Women report a wide range of emotional symptoms, including depression, anxiety, anger, tension and irritability. These symptoms are worse a week or so before a woman's menstrual period, but are generally resolved immediately thereafter.

Many women also suffer from a condition called menorrhagia (heavy bleeding). Menorrhagia is a clinical problem characterized by extremely heavy flow/bleeding (characterized by blood loss exceeding 80 cc/month) and major discomfort. It is estimated that 1 in 5 women between the ages of 35 and 50, or approximately 6.4 million women in the United States alone, are affected by menorrhagia. Fibroids, hormonal imbalance and certain drugs, such as anticoagulants and anti-inflammatory medications, are common causes of heavy bleeding.

Women diagnosed with menorrhagia or dysmenorrhea have limited treatment options available to them. Conventional therapy or treatment options include drug therapy followed by dilation and curettage (D & C), endometrial ablation/resection, and, as a last resort, hysterectomy.

Drug therapy is generally the first treatment option employed to treat excessive bleeding. Birth control pills, progestin, danazol and gonadotropin-releaseing hormone (GnRH) are a few examples of drug treatments prescribed to reduce bleeding. In general, birth control pills contain synthetic forms of estrogen and progesterone, which prevent ovulation and, thereby, reduce endometrial build-up or thickness. As a result, pill users normally have lighter or minimal menstrual bleeding. Progestin, another synthetic form of progesterone, balances the effects of estrogen normally produced by the body and, similar to the pill, reduces endometrial growth. Often, Danazol and other GnRH agents are prescribed to suppress estrogen production and ovulation. As a result, menstrual bleeding stops or is significantly reduced. However, side-effects of such treatments may include bloating, breast tenderness, increased risk of osteoporosis and high cholesterol.

D & C, frequently a second treatment option for excessive bleeding, is a very common, minor surgical procedure that is generally performed on an outpatient basis in a hospital. Usually, the patient is given a general anesthetic, although the procedure occasionally is performed using only a local anesthetic. The dilation step of the procedure involves dilating or stretching the cervix, which is the lower part of the uterus. Once the cervix is appropriately dilated, the curettage step can then be performed. During curettage, a curette (a spoon-shaped instrument) is inserted through the vagina, past the cervix and into the uterus. The curette is then used to scrape the inside surfaces of the uterus to remove the uterine lining or endometrium. Possible problems relating to this procedure include reactions to anesthesia, damage to the cervix or uterus requiring further surgery, infection, or excessive bleeding. In general, most women experience fairly mild cramping after the procedure.

Endometrial ablation has become more popular and has been offered as another alternative treatment to hysterectomy for patients suffering from menorrhagia. In 1996, 179,000 ablation procedures were performed, up from 49,000 in 1993. This technique is intended to permanently ablate all layers of the endometrium and allow the cavity to become lined with fibrous tissue.

In either endometrial ablation or resection, an attempt is made to remove or destroy the entire lining of the uterus (the endometrium). Endometrial resection, first described in 1983 by De Cherney et al., involves the use of a resectoscope-cutting loop to perform endometrial ablation to remove the lining of the uterus. In contrast, ablation generally uses either vaporization, coagulation or some other thermal energy source to destroy the uterine lining. There are various methods by which an endometrial ablation procedure may be performed. These methods include roller ball electrocautery, cryo-cauterization, microwave, free circulating water, vaporization, balloon ablation and photodynamic therapy. In general, these procedures are performed in a hospital or surgery center, not in the physician's office, due to the need for anesthesia.

Although ablation and resection procedures are often discussed as if they are the same, they differ significantly. For example, some physicians argue that resection is more difficult. However, when it is performed skillfully, resection has much better results (control of bleeding in up to 88% of patients) than roller ball ablation (40% to 55%) and newer ablation techniques (3% to 30%).

In general, endometrial ablation is less costly and requires less recovery time for the patient. However, the procedure has received mixed results for controlling bleeding, depending on the technique used, and has a limited success rate of no greater than 20% when defined as complete cessation of bleeding. During one five-year study of 525 women with an average age of 42, endometrial ablation completely stopped uterine bleeding only 26% to 40% of the time. However, approximately 79% to 87% of the women were satisfied with the surgery. About 16% of the women required a repeat ablation to stop bleeding and 9% of the women ultimately opted for a hysterectomy. Research has also shown that the effectiveness of endometrial ablation may decline over years, with menstruation returning in about one-third of women.

It should be noted, however, that the goal of endometrial ablation was never to create amenorrhea (cessation of menstrual periods). This procedure was originally developed as a less invasive alternative to hysterectomy in order to return women with menorrhagia to a normal menstrual flow.

Although ablation and resection procedures are less invasive than hysterectomies, there are various complications that may occur. Examples of possible complications include perforation of the uterus, injury to the intestine, hemorrhage or infection. Another concern associated with ablation treatment involves the risk of cancer. Since ablation does not remove the uterus, women still are at risk for developing endometrial cancer (although the risk is reduced; however, no clinical proof is currently available). Further, because endometrial ablation alters the wall of the uterus, early detection of cancerous changes may be difficult to identify. Additional side-effects, together with low success rates at achieving amenorrhea, associated with ablation and resection procedures cause many women to choose hysterectomy as a preferred treatment option.

Despite its life-altering effects, over 600,000 hysterectomies are performed each year in the United States. It is estimated that 1 in 3 women in the U.S. have a hysterectomy before the age of 65. Menorrhagia is the most common reason why hysterectomies are performed. Several studies have estimated that menorrhagia is the cause of 30% (some studies as high as 50%) of the 600,000 annual hysterectomies, resulting in a basis of 180,000 to 300,000 procedures annually. Financially, these numbers translate into annual hospital costs that exceed $5 billion per year.

Based on these statistics, hysterectomy is a very common operation. In general, there are three types of hysterectomies: partial, total and radical. As shown in FIG. 1, a partial hysterectomy involves removal of the upper portion 10 of the uterus 12 (whereby the dotted lines in the figure indicate the area removed), leaving the cervix 14 and the base 16 of the uterus 12 intact. FIG. 2 illustrates a total hysterectomy whereby the entire uterus 12 and cervix 14 are removed. A radical hysterectomy, shown in FIG. 3, entails removal of the uterus 12, both Fallopian tubes 18, both ovaries 20, and the upper part of the vagina 22. Each of the above three procedures may be performed via an abdominal incision (abdominal hysterectomy) or through a vaginal incision (vaginal hysterectomy).

After the operation, the hospital stay is generally less than a week, depending on the type of hysterectomy and whether there are any complications. Since a hysterectomy is a major operation, discomfort and pain from the surgical incision are most pronounced during the first few days after surgery. Medication is available to minimize these symptoms. By the second or third day, most patients are up walking. Normal activity can usually be resumed in four to eight weeks and sexual activity can usually be resumed in six to eight weeks.

Both hysterectomy and endometrial ablation/resection eliminate the possibility of childbearing. In addition, hysterectomy requires a lifetime of hormone therapy when the ovaries are removed. As such, the long-term risks associated with these procedures are quite high and may lead to other more serious complications, such as mixed mesodermal tumors or uterine cancer.

In view of the above, there is a need for a minimally invasive device and method to treat abnormal intrauterine bleeding. In particular, it is desirable that the device have a high success rate at treating menorrhagia and have minimal to no side-effects or related complications. Such a device must also be biocompatible and non-toxic. In addition, the related treatment methods should reduce patient recovery times and hospital costs. Overall, the method of treatment should also improve the quality of life for patients.

### BRIEF SUMMARY OF THE INVENTION

The present invention contemplates a method of creating tissue fibrosis in a body cavity. In general, the method comprises inserting an implantable device within the body cavity. The method also includes locating the implantable device at an optimal site within the body cavity, wherein the optimal site promotes effective fibrosis or scar tissue formation to control bleeding.

### BRIEF DESCRIPTION OF THE DRAWINGS

Other features and advantages of the present invention will be seen as the following description of particular embodiments progresses in conjunction with the drawings, in which:

FIG. 1 is a sectional view of an embodiment of a hysterectomy;

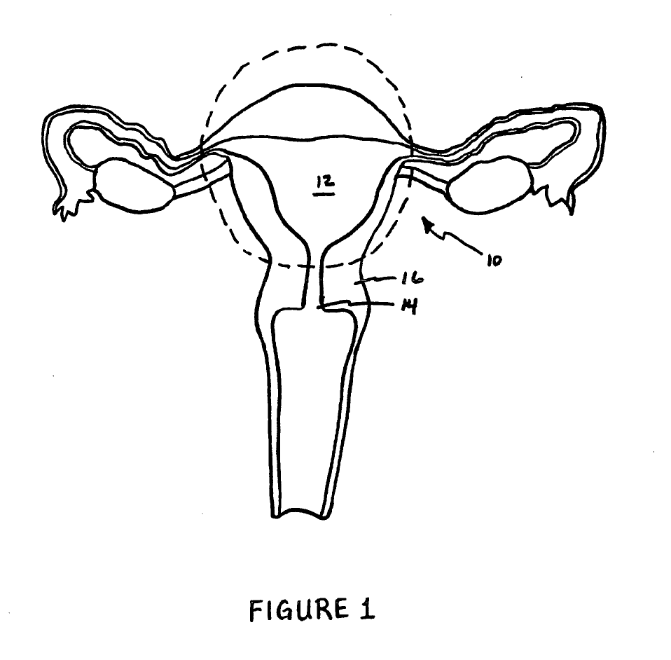

FIG. 2 is a sectional view of another embodiment of a hysterectomy;

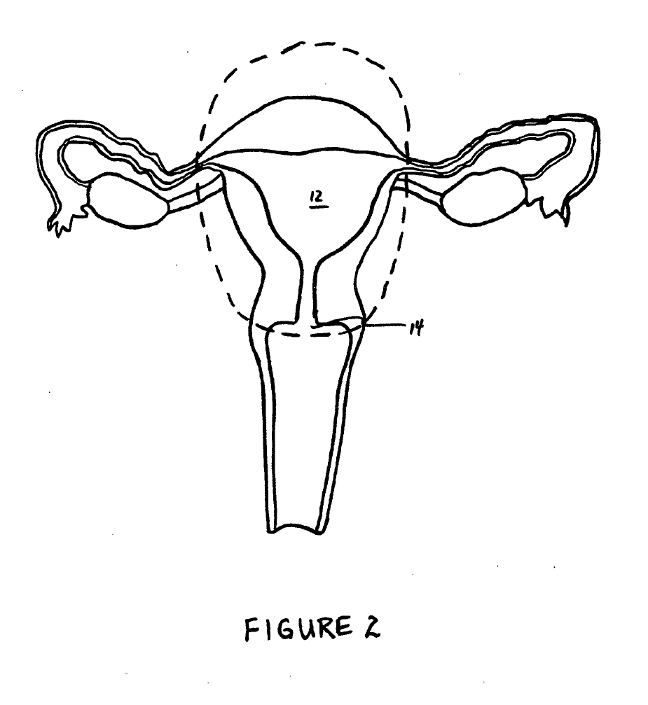

FIG. 3 is a sectional view of yet another embodiment of a hysterectomy;

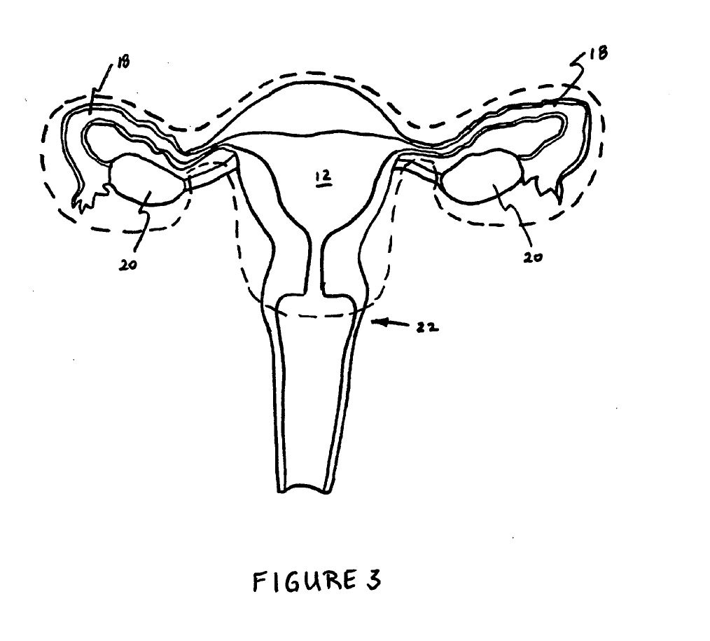

FIG. 4 is a sectional view of an embodiment of a device implanted within a uterus in accordance with the present invention;

FIG. 5A is a front, sectional view of a uterus;

FIG. 5B is a side, sectional view of a uterus;

FIG. 6 is a sectional view of one embodiment of an implant device in accordance with the present invention;

FIG. 7 is a perspective view of another embodiment of an implant device in accordance with the present invention;

FIG. 8 illustrates a perspective view of an alternate embodiment of an implant device in accordance with the present invention; and

FIG. 9 illustrates another view of an alternate embodiment of an implant device in accordance with the present invention.

### DETAILED DESCRIPTION OF THE INVENTION

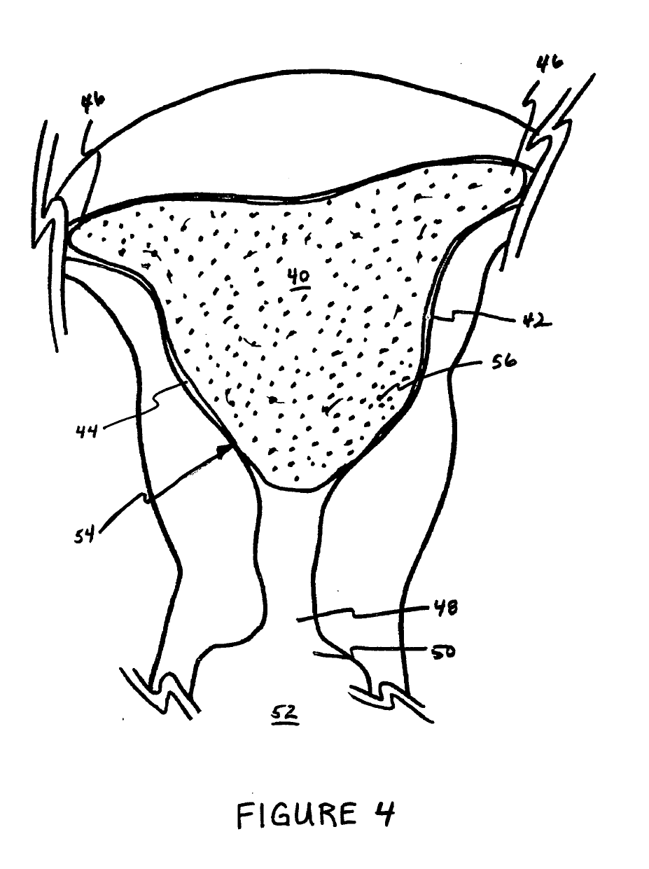

Referring to FIG. 4, an embodiment of the intrauterine implant device 40 in accordance with the present invention is shown deployed within a uterus 42. Although the invention as disclosed herein generally refers to a uterus, other body cavities, such as the endocervical canal, the lower uterine segment and cavities within a heart, abdomen or other similar cavities, are also included within the scope of the present invention.

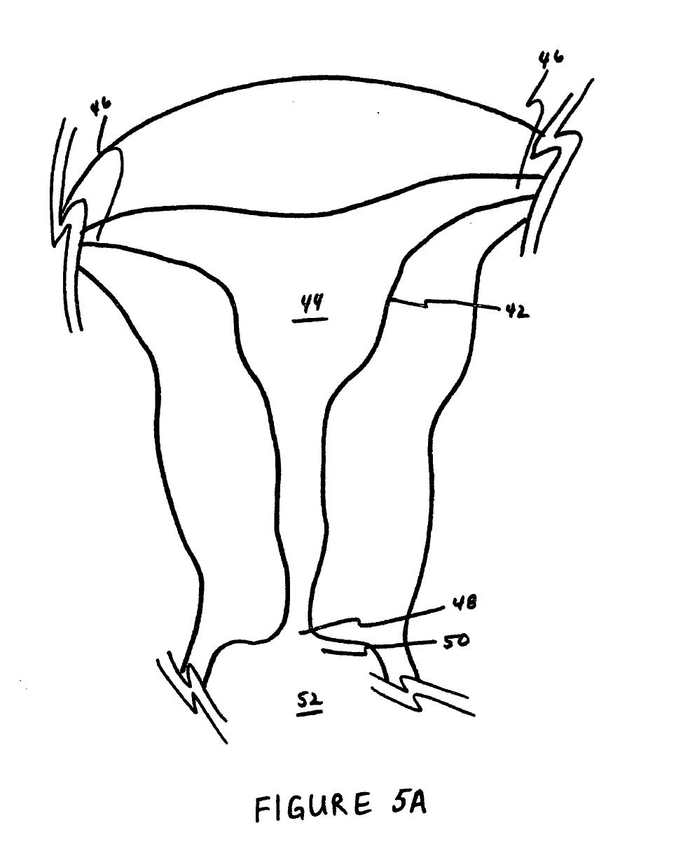

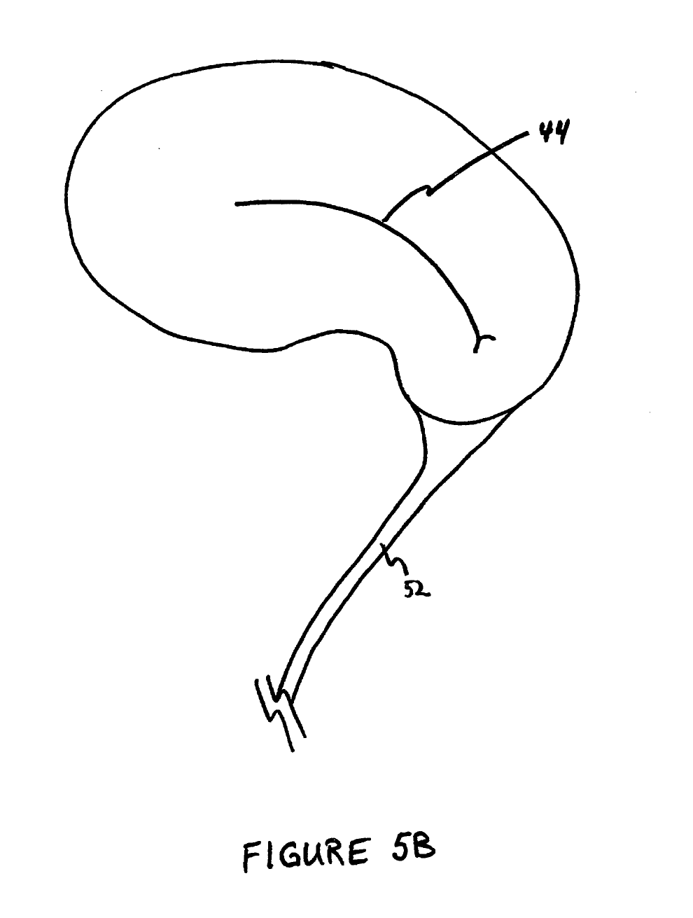

The uterus 42, or womb, is part of the female internal genitals. The uterus 42 is a hollow, muscular organ approximately four inches long and three inches wide and is generally shaped like an upside-down pear. It should be noted that the uterus 42 depicted in FIG. 4 is in a distended state to clearly show the uterine cavity 44. However, it is understood that the uterine cavity 44 is normally in a collapsed state, as shown in FIGS. 5A and 5B.

Two openings 46 located at the upper end of the uterus 42 lead to the Fallopian tubes that are connected to the ovaries (not shown). Opposite to the upper end openings 42 is a lower, narrow open end 48 that forms the cervix 50 of the uterus 42 and extends to the vagina 52. The thick walls of the uterus 42 are comprised of three layers of tissue and muscle: the inner endometrial layer, the middle myometrial layer and the outer perimetrial layer. It is the inner endometrial layer or lining that separates from the uterus 42 and leaves the body as the menstrual flow during a woman's menstrual period.

Excessive menstrual flow or bleeding, termed menorrhagia, is indicative of abnormal sloughing of the endometrial tissue layer. Unlike conventional therapies such as hysterectomy or ablation/resection procedures, as described above, the device 40 of the present invention achieves amenorrhea (i.e., cessation of bleeding) by way of an implant and/or substance that promotes an inflammatory response ultimately resulting in fibrosis.

Fibrosis refers to the development of fibrous scar tissue or adhesions and typically occurs when the normal processes involved in tissue repair get out of control, a process termed fibrogenesis. Fibrogenesis results when tissue trauma or a fibro-inductive substance applied to a target site stimulates inflammatory cells that release cytokines and other chemicals which cause cells, known as fibroblasts, to form around the target site and synthesize fibrous tissue. In particular, collagen, various glycoproteins, and other components of connective tissue that make up the extracellular matrix (the structure between cells) in healthy tissues proliferate excessively in fibrotic tissue. It is believed that it is the excessive cell proliferation (i.e., tissue fibrosis or scar tissue) that causes a deactivation of the endometrial tissue resulting in cessation of bleeding. Other modulating factors, not specifically described herein, but included within the scope of the claimed invention, may also contribute to the deactivation of the endometrium. It is important to note that the endometrial tissue is deactivated through means other than the direct destruction of the lining, and that endometrial deactivation may be seen even in the presence of minor tissue fibrosis.

As shown in FIG. 4, one embodiment of the device 40 of the present invention used to promote tissue fibrosis includes a porous bag or container 54 that is deliverable within a body cavity, such as a uterus. The bag 54 is preferably fabricated from an elastic and/or expandable material that generally conforms to the internal structure of the uterine cavity 44. Bag expansion is accomplished by filling and packing the bag 54 with a fibro-inductive component 56 configured, for example, as beads. When in its expanded state, the bag 54 exerts pressure on the surrounding tissue and irritates the endometrial tissue, possibly as deep as the myometrium. This, in turns, causes fibrosis within the uterus and, subsequently, cessation of bleeding.

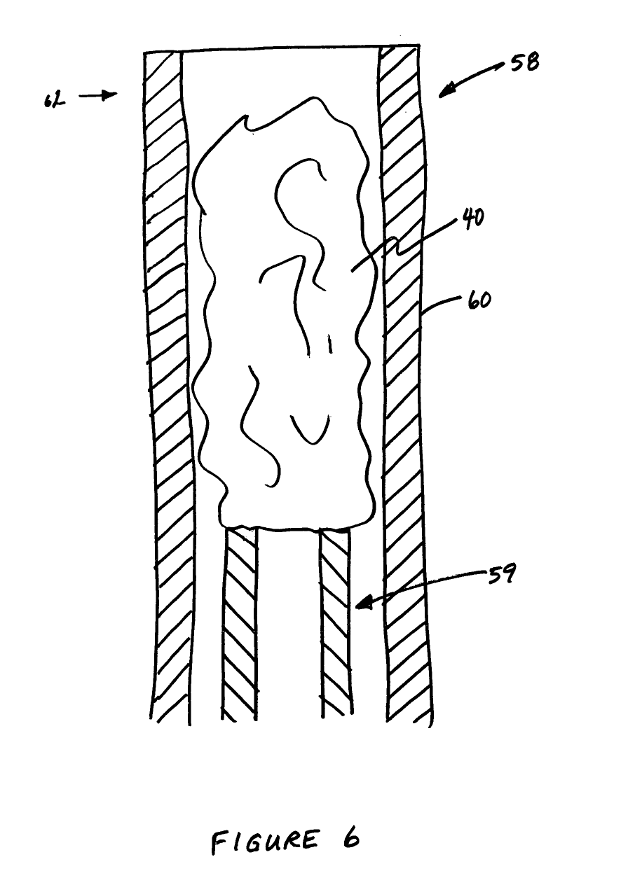

Device delivery may be accomplished using a catheter, cannula or other similar type of delivery tool 58. As shown in FIG. 6, the implantable device 40, including a second cannula 59 in fluid communication with and removably attached to the device 40, is housed within the delivery tool 58. Preferably, the outer surface 60 of the delivery tool 58 is smooth and/or lubricious to allow for easy insertion into the patient (not shown).

During the delivery procedure, the delivery tool 58 is inserted transcervically into the patient and the distal section 62 is positioned within the uterus 42 (not shown). The implantable device 40 is then deployed through the delivery tool 58 and maneuvered to the target site. Once properly positioned within the uterus 42, the device 40 is filled and packed with a fibro-inductive material (not shown) that is passed through the second cannula 59 and dispensed into the interior of the device 40 using a stylet, syringe or other similar tool. After the device 40 is properly filled, the second cannula 59 is rotated to seal and subsequently disconnect from the device 40. Both the delivery tool 58 and second cannula 59 are removed from the patient at the conclusion of the delivery procedure.

The method of filling/loading the beads/fibro-inductive component 56 into the bag 54 is similar to the method by which insulation is blown into an attic or other open space. As such, the fibro-inductive component 56 can be configured as atomized micro-particles, semi-rigid foam, suspended aggregate, particulates, powder or other similar forms, including combinations thereof. Alternatively, the fibro-inductive component 56 may be suspended in a liquid, gas, foam or other flowable substance capable of expanding the device 40 in the uterus 42. In one embodiment, the particular composition or material make-up of the flowable substance is such that its viscosity can be modified through thermal changes. The thermal changes may include those produced externally or generated by the patient's own body temperature. One example of a thermally-sensitive material is a polymer substance. However, it should be noted that other thermally-sensitive materials not specifically disclosed herein, but well known in the art, may also be used with the present invention.

The resulting pressure exerted by the expanded device 40 and/or the fibro-inductive material that is dispensed via the porous device 40 into the endometrium cause a persistent irritation and inflammatory response that, ultimately, lead to fibrosis. This, in turn, promotes a deactivation of the endometrial tissue resulting in cessation of bleeding.

Both the bag 54 and the fibro-inductive component 56 of the device 40 of the present invention can be made of a variety of materials. Examples of these materials include, but not limited to, mesh, suture, gel, porous, allograft, protein, hydrogel, collagen, spun fibers, bone particulate, cellulose, alginate, tissue, kitosan, particulate, composite, aggregate, foam and any combination of materials. The properties or characteristics of these materials may be non-absorbable, temporary/absorbable (whereby the material is broken down by the body through any means including enzymatic, hydrolytic, mechanical, etc. and excreted), or permanent/resorbable (whereby the material is remodeled through some process to form host or other similar tissue). In addition, the device material should be biocompatible, non-toxic and, preferably, one that is approved/cleared by the Food and Drug Administration (FDA). Further, it is desirable that the material be capable of conforming to irregular volumes and/or shapes to exert sufficient pressure on the surrounding tissue and, possibly, irritate tissue deep within the myometrium. In general, the device 40 should be designed such that it can be placed in, stored in and deployed from a catheter or similar device delivery tool.

In one embodiment, the device material is fabricated from a woven, surgical mesh. Alternatively, the mesh can be braided, spun, knitted, non-woven and any structural combination thereof. Examples of representative surgical meshes include GORE-TEX® (manufactured by W. L. Gore & Associates, Arizona), Marlex® (manufactured by C. R. Bard, New Jersey), Mersilene® (manufactured by Johnson & Johnson, New Jersey), Prolene® (manufactured by Johnson & Johnson, New Jersey), Surgipro® (manufactured by US Surgical, Connecticut), Surgisis® (manufactured by SIS Technology Cook Group, Indiana), Vicryl® (manufactured by Johnson & Johnson, New Jersey) and Atrium Surgical Mesh (manufactured by Atrium, New Hampshire) and Dacron®. Specific references for these materials may be found in the manufactures' product catalogues. Additional surgical mesh materials such as polyester, felt, adhesion barrier materials, adhesion promoter materials, polyethylene fiber, non-absorbable mesh, PTFE (Polytetrafluoroethylene), absorbable mesh and other mesh materials not specifically disclosed herein may also be used.

In another embodiment of the invention, the implant 40 is made of a woven material, such as a fabric with a specific weave that is also biocompatible. In this configuration, the material of the device creates a lattice-like structure (having openings or pores) which releases the fibro-inductive component into surrounding tissue, resulting in fibrous tissue formation. The material may be metallic, polymeric or a bio-material (including combinations of materials) and can be absorbable or non-absorbable, depending on the physical and procedural requirements. Additional material specifications or variables may include type of weave (such as plain, open, closed, twill, dutch, reverse dutch, twill dutch, or taffeta, including combinations of weaves), mesh count, fiber diameter, filament type (such as monofilament fiber or multi-filament fiber) or whether there are interconnection of weave points.

Alternatively, the device 40 of the present invention can also be made of non-woven materials. One type of non-woven material is a random fiber bundle. The fiber bundle may be a thin mat, similar to a woven mesh, with an irregular fiber pattern. Examples of materials having an irregular fiber pattern include Scotchbrite® or Brillo® pad materials. In addition, the material may be fabricated from any monofilament or multi-filament material. An example of a monofilament material that can be used for the implant is suture material, such as Prolene® or Vicryl® (manufactured by Johnson & Johnson, New Jersey). Although the fibers of the non-woven material are arranged in a random orientation, the configuration of the fibers produces an associated effective pore size. Additional examples of non-woven materials include all the materials listed above, since materials fabricated into a woven product can also be manufactured into a random fiber bundle.

In another embodiment, the device 40 of the present invention is made of porous materials. Examples of such porous materials include, but are not limited to, ceramics, alumina, silicon, powdered metals, Nitinol®, stainless steel, titanium, porous polymers, such as polypropylene, polyethylene, acetal, nylon, polyester, and any combination of such materials. Although these materials (and others not specifically described, but included in the scope of the claimed invention) may not be inherently porous, various manufacturing and processing techniques may be used to achieve the desired porosity characteristics.

In another embodiment of the invention, the device or implant 40 is fabricated from a liquid based component, such as collagen, tissue/collagen, thrombin, polymer and fibrin-based sealants, including combinations thereof. In general, these materials are typically configured in a liquid format. However, collagen is a very common substance and may be found in numerous configurations, including flour, compressed mat pad, non-woven fiber or other molded, extruded or compressed shapes with varying density and/or porosity. Examples of collagen and tissue/collagen materials contemplated herein include Avitene® (manufactured by C. R. Bard, New Jersey), Helitene® (manufactured by Integra LifeSciences Corporation, New Jersey), Dermalogen®, Dermaplant™ (manufactured by Collagenesis, Inc, Massachusetts), Apligraf®, Engineered Collagen Matrix™ and Vitrix™ (manufactured by Organogenesis Inc., Massachusetts). The collagen may be synthesized or derived from bovine, porcine or human sources.

An example of a collagen-thrombin sealant that may also be used with the present invention is Costasis®. Costasis®, manufactured by Cohesion Technologies, California, is a collagen-thrombin composite for use as a hemostatic agent to arrest or control bleeding at various sites within the patient's body. This material is comprised of bovine fibrillar collagen and bovine thrombin suspended in calcium chloride. At the time of application, fibrinogen (taken, for example, from the patient's plasma) is mixed with the Costasis®, thereby providing fibrinogen that is cleaved by the thrombin to form a collagen-reinforced liquid hemostat. The resultant liquid material may then be applied to the target site to control bleeding.

Alternatively, the physical properties of the liquid sealants may be altered to create hemostatic solids of specific shapes or pliable geometries. In one embodiment, the sealant material may be placed in a carrier matrix that has specific flow requirements and may be activated by heat or moisture to change the sealant's physical characteristics. An example of an appropriate carrier matrix is thrombin-based CoStop®, also manufactured by Cohesion Technologies, California. However, unlike Costasis®, CoStop® does not require plasma from the patient. Simply combining the patient's blood with the thrombin-based CoStop® is sufficient to cause platelet activation. As soon as the combination of blood and thrombin causes platelet activation, the thrombin further catalyzes the mixture to form a fibrin clot. As such, platelet activation initiates clot formation. A collagen-fibrin matrix develops, forming the basis or support-structure for the tissue that will be created at the target site. Thus, when used to treat menorrhagia, CoStop® is placed within the uterus 42 of the patient and forms the collagen-fibrin matrix, which promotes amenorrhea.

In another embodiment, the device 40 of the present invention is made of allograft materials (i.e., a graft of tissue taken from a donor of the same species as the recipient). These materials use the structure and properties of the allograft tissue as a matrix for new tissue formation. Osteofil™ (manufactured by Regeneration Technologies Inc., Florida) is an example of one such material. The Osteofil® is placed within the uterus 42 of the patient and fibrous tissue is formed within the matrix. The allograft tissue from Regeneration Technologies Inc. is initially contemplated as de-mineralized bone; however, other tissues derived from animals or humans may also be used. In addition to Osteofil®, other similar materials including, but not limited to, Natural Matrix (Xenograft), such as OsteoGraf® N-Block (manufactured by Cera Med Dental, LLC, Colorado) and other tissues available from various accredited tissue banks are also within the scope of the claimed invention.

In yet another embodiment, protein materials are used to fabricate the device 40 of the present invention. Various companies and organizations have studied the use of proteins for creating both non-stick and attachable surfaces. One such company is Protein Polymer Technology located in San Diego, Calif. Protein Polymer Technology creates synthetic genes using recombinant DNA technology. In particular, Protein Polymer Technology is able to configure small protein building blocks into high molecular weight polymers.

Another company that uses proprietary technology to create application specific proteins is Gel-Del Technologies (St. Paul, Minn.). Gel-Del Technologies, like Protein Polymer Technology, and other similar companies process proteins using various methods. The physical structure and composition of the protein are modified to create a wide variety of properties for the protein. The physical characteristics (for example, shape) of the protein and its side chain elements influence the development of a fibrous response. In particular, the available side chain elements regulate selective infiltration of tissue into the protein structure, thereby producing fibrosis at the tissue target site.

In general, proteins may be developed into a wide variety of formats. Examples of various protein formats include small beads, sheets, strips, tubes, cylinders or other regular or irregular shaped configurations. The protein format allows the protein to be implanted in, for example, the uterus 42 to create the response necessary for fibrosis.

In another embodiment of the invention, the device or implant 40 is fabricated from hydrogel materials. Hydrogels are coherent three-dimensional polymeric networks that can absorb large quantities of water without dissolution of the polymer network. Classes of hydrogels, based on their method of preparation, include homopolymer hydrogels, copolymer hydrogels, multipolymer hydrogels and interpenetrating hydrogels. In general, hydrogels are hydrophilic polymers incorporating Chitson derivatives or polyethylenimine together with polyvinylpyrrolidone (PVP). Hydrogels may also include cellulose derivatives, polyvinyl alcohol (PVA) or polyethylene glycol (PEG). An example of one common hydrogel is polyHEMA (poly(2-hydroxyethyl) methacrylate). These highly compatible water-soluble polymer systems naturally combine with each other to form gels possessing excellent physical properties. These properties may be varied by the chemistries of the gel (i.e., compounding), active ingredients and biomolecules, which can be readily incorporated without impairing biological activity. Virtually any material that can be dissolved, emulsified, or suspended can be added prior to gel-formation and evenly distributed in the finished gel.

The hydrogel Aquatrix™ II (manufactured by Hydromer, New Jersey) is an example of one such hydrogel product. The gel may be loaded with any of the above-mentioned materials, such as Marlex® (manufactured by C. R. Bard, New Jersey), Mersilene® (manufactured by Johnson & Johnson, New Jersey), Surgipro® (manufactured by US Surgical, Connecticut), Surgisis® (manufactured by SIS Technology Cook Group, or any other material that is pulverized, ground, etc. and combined with the hydrogel material. In this configuration, the hydrogel is acting as a carrier material to allow for dispensing of the scaffold or lattice material. The material can then be delivered as a flowable liquid with a suspension of particles. Further, the gel may be formulated to be absorbed or resorbed by the body within 30 to 60 days. In an alternate embodiment, the gel may be formulated to be non-absorbable. In the case of a non-absorbable gel, the gel may be placed at the target site and then blown with a gas to form small pores. The pores function in a manner similar to the mesh openings or pores, allowing release of the fibro-inductive component into surrounding tissue, resulting in fibrous tissue formation.

In general, the materials used with the device 40 of the present invention may be comprised of a combination of absorbable and/or non-absorbable materials or components. In one embodiment, described in further detail below, the absorbable material may be comprised of a radio-opaque marker, or any other type of imagable marker, that allows the target site to be imaged. In another embodiment, the absorbable material may be used to fixate the non-absorbable material at the target site in the patient.

Preferably, the size and/or configuration of the device 40 is optimized to promote fibrosis or scar tissue development within the uterus 42. In one embodiment, the device 40 is configured to contact substantially the entire area of the endometrium to maximize the amount (i.e., up to 100% coverage) of fibrosis within the uterus 42. Alternatively, there may be optimal locations within the uterus 42 for site-specific deployment and/or placement of the device 40. As such, the implant 40 need only contact specific or discrete areas of the endometrium for effective fibrosis (i.e., fibrosis in less than 100% of the endometrium). For example, the device 40 may be positioned at a specific site only within the uterus 42. Alternatively, a combination of uteral and cervical locations may be used for beneficial fibrosis.

As another example, the device 40 may be located in the cervical canal or lower one-third portion of the uterus to control bleeding. The significance of this is that the lower one-third of the uterus is uniform in shape and, therefore, more easily treated compared to the entire, irregularly shaped cavity. In addition, by treating only the lower one-third of the uterus, there is less trauma to the patient, less required material or energy, and a technically easier procedure with easier access to the target site.

Physiologically, the tissue in the lower one-third of the uterus is different and has been reported to not cycle like the rest of the uterine cavity. It has also been reported that there is a greater concentration of ganglion in the lower one-third of the uterus. As such, the mechanism which controls the lower one-third of the uterus may be a neuro-modulating effect, a signaling phenomenon, a pressure gradient effect, or an evolutionary protection mechanism to protect the species, such that if the lower one-third of the uterus is injured or blocked, bleeding in the upper two-thirds of the uterus may cause hematometria or trapped blood. Thus, in one embodiment of the invention it is only the lower one-third of the uterus that needs to be treated and/or controlled to mitigate and/or eliminate menorrhagia.

Scar tissue formation or coverage is important not only in placement of the coverage (which is related to device placement) but also percentage of coverage. Although the device 40 and methods referenced herein are directed at creating 100% coverage of scar tissue over the entire area of the endometrium, it should be understood that alternative device configurations and methods of use relating to less than 100% endometrial area coverage are also contemplated herein. For example, in general, it is believed that the percent of coverage must be around 75% or greater and/or the placement of coverage should be within the lower one-third of the uterus and/or the entire cervical canal. Other coverage options, though not specifically described herein, are also included within the scope of the claimed invention.

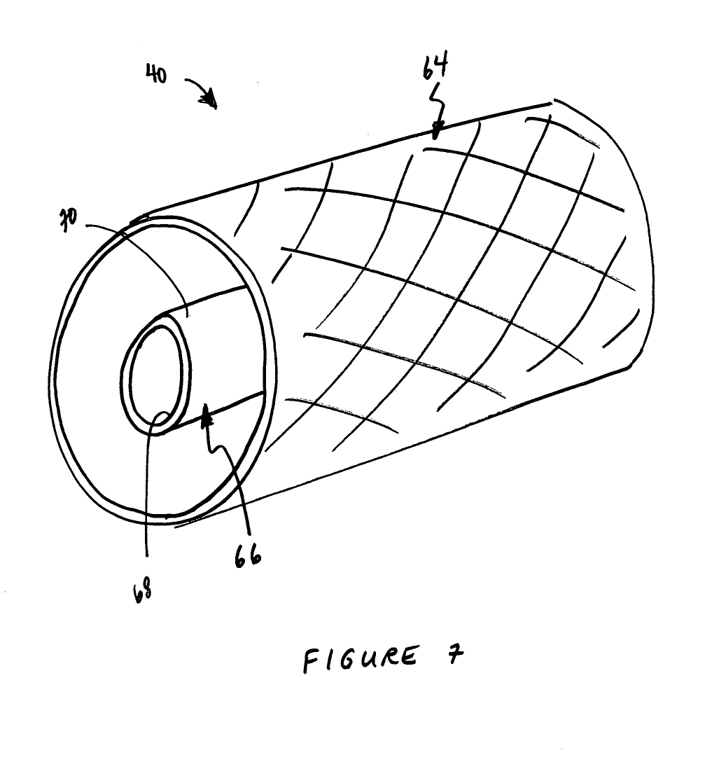

In an alternate embodiment of the present invention, shown in FIG. 7, the intrauterine implant device 40 includes an outer element or stent 64 having an internal access lumen 66. The outer element 64 may be a true stent (such as a Nitinol stent) or other similarly configured component having elastic/memory characteristics and/or properties. Examples of such similarly configured components include, but are not limited to, a polymer material that is rolled and formed into a tubular configuration, a substance that is connected to or surrounds the access lumen 66 (such as a prolene mesh or other biocompatible material), a material that is rolled and/or coiled into a tubular shape, a polymeric substance that covers or is molded over the access lumen 66, a PGA (polyglycolide)/PLA (polylactide) extruded tube that is laser cut or etched to create an expanding lattice, a coiled wire or a coiled spring.

The expansion or memory characteristics of the outer element 66 allow the device 40 to exert pressure and/or trauma against the surrounding tissues (e.g., endometrium) and, in some instances, penetrate into the myometrium, thereby producing a persistent irritation and inflammatory response that ultimately leads to the desired fibrosis. In this regard, the outer element 66 may be formed in a variety of configurations that best conform to the shape of the target site or target area. Examples of outer element configurations include, but are not limited to, cylindrical, tubular, bell-shaped and triangular.

Referring to FIG. 7, the access lumen 66 of the stent 64 is sufficiently sized to allow passage of a hysterescope therethrough. For example, in one embodiment, the access lumen 66 is sized to accommodate a 3-5 mm diameter hysteroscope. In general, the access lumen 66 is configured to allow for drainage of and access to the uterine cavity for diagnostic or therapeutic treatment and procedures. Additional uses of the access lumen 66 include, but are not limited to, dispensing fluids, creating vacuum, delivering tools and deploying an implant within the uterus. In addition, the pathway of the access lumen 66 may also be used for post-procedure therapy or future diagnosis of the uterine cavity. For example, if required, a biopsy of the uterine tissue may be performed using the lumen/channel as an access port.

A removable plug or cap (not shown) configured to plug or occlude one or both ends of the access lumen 66 may also be used with the device 40 of the present invention. In this regard, the cap functions to permanently or temporarily contain fluids, gels, or other substances within the access lumen 66. Alternatively, the cap may be used to plug or occlude the outer element 64 either in addition to or separate from the access lumen 66.

A variety of materials may be used to fabricate the outer element 64 and access lumen 66 of the present invention. In one embodiment, the access lumen 66 is fabricated from a non-resorbable, biocompatible material, such as Teflon, nylon, silicone polyurethane, polypropylene, stainless steel, or Nitinol. In addition, the inside surface 68 and/or outside surface 70 of the access lumen 66 may be coated with an adhesion-preventing material. In an alternate embodiment, the outside surface 70 may be coated with a fibro-inductive material or coating. In yet another embodiment, the outer surface 72 of the outer element 64 may be textured and/or coated with one or more materials (e.g., chemicals, drugs, etc.). Further, the outer element 66 may be filled with an expandable material, such as collagen, to cause device expansion and/or tissue in-growth. In another embodiment, the outer element 66 and/or lumen 64 may be filled with a fibro-inductive material, a sclerosis agent or other substance known to incite a reproducible inflammatory response. Alternatively, the outer element 66 may have a self-expanding structure attached to its perimeter to motivate it to unfold. This structure may consist of a material that has a memory and/or spring-like structure or behavior (i.e., elastic properties). Examples of representative materials include, but are not limited to, metallics, such as Nitinol® or stainless steel, and polymerics, such as nylon, acetal or propylene. Other materials, fillers and/or coatings, such as those previously disclosed and others not specifically listed herein, are also included within the scope of the claimed invention.

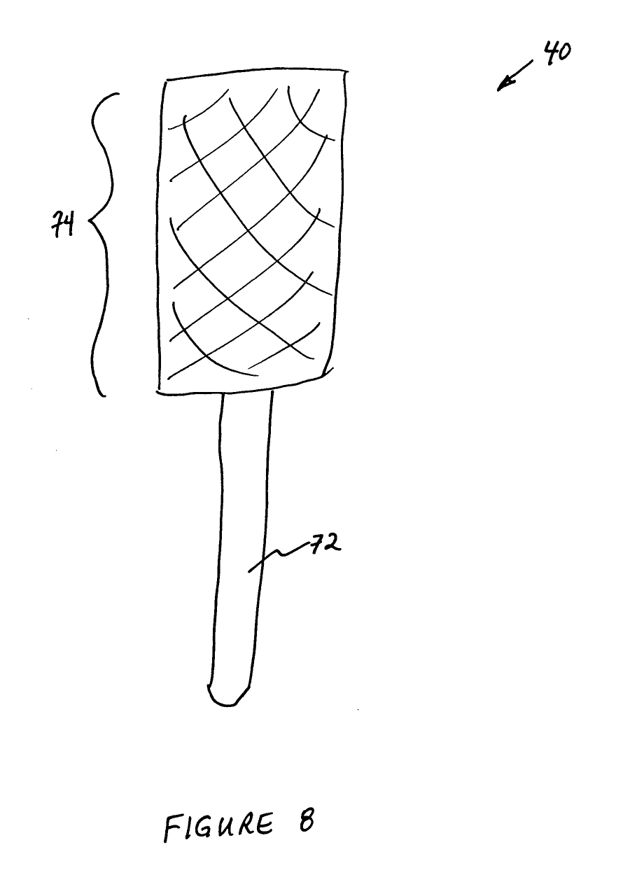

FIG. 8 shows an alternate embodiment of the invention in which a rigid handle or stylet 72 is attached to the implant device 40. During the delivery and/or treatment procedure, the device 40 is inserted transcervically into the patient and its distal section 74 is positioned within the uterus 42 (not shown). Once properly positioned, the device 40 is activated to adequately expand the distal section 74 at the target site. The device 40 is then further manipulated, for example, rolled, twisted, pushed and/or pulled, so that the distal section 74 causes sufficient insult to the target tissue. Next, the distal section 74 of the device 40 is once again positioned at the target site or implant location within the uterus. The handle 72 is then disconnected so that only the distal section 74 of the device 40 remains in the uterus 42 of the patient. The tissue trauma produced from device manipulation and the pressure exerted by the expanded device 40 promotes growth of fibrous tissue, which ultimately causes cessation of bleeding.

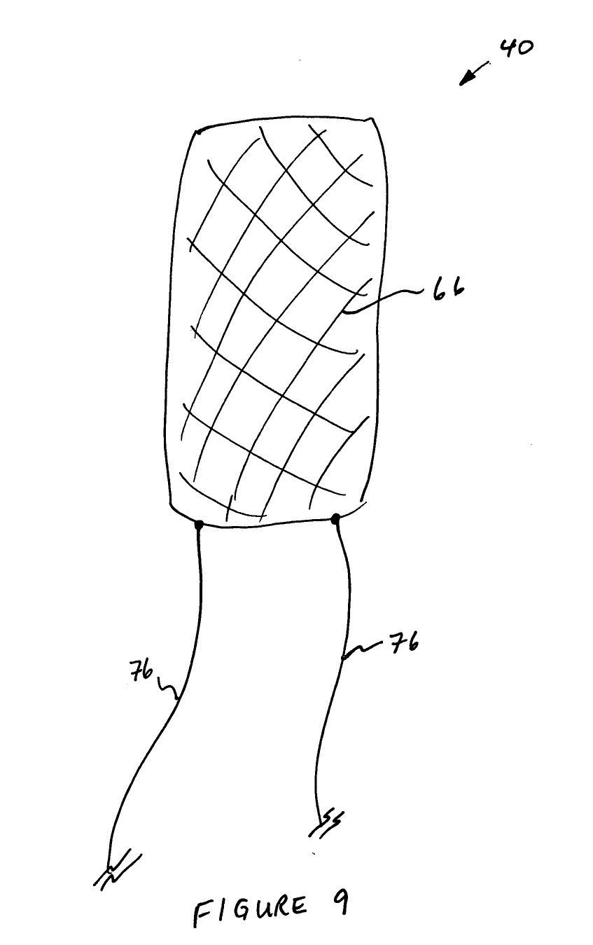

In another embodiment of the present invention, illustrated in FIG. 9, the outer element 66 of the intrauterine device 40 is connected to a generator or power supply (not shown). In this regard, one or more leads or wires 76 from an energy source are connected to the outer element 66 and/or access lumen 64 (not shown) of the device 40 for conducting energy from the energy source to the implanted device 40. The leads 76 may be either permanently or removably connected to the device 40 depending on the type of procedure being performed.

Once the device 40 is properly positioned and deployed at the target site, the energy source is activated. In this configuration, the implant device 40 acts as a resistor and heats tissue at the target site to a predetermined depth and temperature. Alternatively, energy applied to the device 40 may be used to cool tissues to a desired temperature and depth. The resulting tissue trauma from heating and/or cooling the target site promotes the development of fibrous tissue and, ultimately, amenorrhea. A variety of energy sources may be used to heat and/or cool the device 40 including, but not limited to, RF, DC, microwave, and laser.

In an alternate embodiment, an applied electric field is used to drive charged molecules (i.e., ions) of a selected material, drug, agent, substance, fluid, gel or chemical of the device 40 into the tissue target site. This process, known as iontophoresis, controllably delivers the selected material to a predetermined depth of tissue penetration. As previously described, the selected material may then necrose, scierose or cause other interaction with the uterine tissue and create the desired fibrosis. Examples of materials or substances, either alone or in combination, that can be used with the device of the present invention include, but are not limited to, anesthetics, silver nitrate, tetracycline, and ethanol.

Another embodiment of the present invention uses drugs, hormones or other chemicals either alone or in conjunction with the implant devices 40 previously disclosed. For example, the devices 40 are coated with chemicals configured in a dry format. The chemicals are hydrolyzed and, thereby, activated when they come in contact with the patient's body fluids and/or tissues. Alternatively, the chemical(s) are dispensed in a liquid format at the treatment site and allowed to act upon the tissue for a specified time period. At the end of the time period, the implant 40 may be removed or, as an alternative, the reaction is stopped prior to the implant 40 being removed. Examples of appropriate chemicals include weak acids, weak bases, saline (with a high concentration of salt to create an osmotic effect), silver nitrate, quinine solution, sodium morrhuate, sodium tetrade, alcohols, alcohols with formalin (i.e., formaldehyde) and other similar sclerosing/necrosing agents or chemicals that cause insult/trauma to the endometrium and/or myometrium.

In an alternate embodiment, the device 40 is coated with one or more chemicals, drugs or other substances that form a brittle coating on the external surface of the device 40. As such, expansion of the device 40 during device deployment causes the coating to crack and/or break off of the external surface and cover the tissue target area. Subsequent exposure to moisture from the patient's body fluids and/or fluid dispensed at the target site activates the coating, which promotes growth of fibrous tissue and ultimately causes cessation of bleeding.

In addition to reducing and/or eliminating menorrhagia, the device 40 of the present invention can also be used as a uterine marker. The marker provides the physician with the ability to visualize and quantify any endometrial growth or abnormality, such as endometrial hyperplasia and/or endometrial cancer. In this regard, the marker may be used as an absolute reference from which the physician may gage the difference of other features (growths or other irregularities). The marker device 40 of the present invention may also be used to assist the physician in determining the plane or location of view (e.g., determines the depth of the imaging plane) such that the cross-section or outside/inside diameters of the uterus may be determined and compared with subsequent diagnostic procedures. The marker device 40 may also be used by the physician when performing a non-invasive biopsy, using the marker as a landmark for guidance to the site under an imaging technique. Therefore, the marker acts as a landmark to assist the physician in determining visual or dimensional differences in the uterus.

In general, the marker component is biocompatible and stable when embedded or implanted over long periods of time (i.e., permanently) within tissue formed on the interior of the uterus 42. As such, the marker material should have good dimensional stability and allow for visualization when imaged using ultrasound, magnetic resonance imaging (MRI), computed tomography (CT), x-ray or other common imaging technique, including any combination of such techniques. The marker can be incorporated into the implant device 40 or can be provided as a stand-alone device.

When combined with the implant device 40, the marker allows the physician to determine placement of the implant device 40 (i.e., coverage, position, etc.), both short term and long term, and track/assess changes in the surrounding tissue. In one embodiment, the fibrosis promoting substance of the device 40 is connected to the marker (which may be configured as a bead or other configuration) to ensure that the marker remains in a fixed, known location. In another embodiment of the invention, the marker is suspended in the fibrosis promoting substance of the device 40.

Although only one marker is sufficient for a variety of diagnostic procedures, multiple markers may also be used. In particular, multiple markers may have the added benefit of allowing for more exact measurement or better visualization, depending on the placement of the marker to the area of interest.

Regardless of the exact form, the uterine marker greatly aids in the early detection of uterine cancer or other abnormalities, and offers a major benefit not available with conventional diagnostic techniques or procedures. With the uterine marker, any physician can easily and quickly evaluate the patient and image and measure the uterine marker locations and related attributes (such as distances between marker components) using conventional imaging equipment.

In addition to providing an effective means of treating uterine disorders, the device and method of use of the present invention effectively reduce pain, infections and post operative hospital stays. Further, the various treatment methods also improve the quality of life for patients.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An intrauterine implant comprising:
a shape conformable substance connected to a delivery device;
said shape conformable substance sized and shaped for placement in the uterus of a patient; and,
said shape conformable substance containing a tissue insult mechanism, said tissue insult mechanism being sufficiently prominent in said shape conformable substance such that insult on uterine tissue from said mechanism substantially alleviates undesirable uterine bleeding.

2. An implant according to claim 1, wherein said shape conformable substance is a flexible container deliverable into said uterus.

3. An implant according to claim 2, wherein said tissue insult mechanism in said flexible container is a fibro-inductive agent.

4. An implant according to claim 3, wherein said fibro-inductive agent is selected from the group consisting of: beads, spun fibers, bone particulate, cellulose, alginate, tissue, kitosan, particulate, composite, aggregate, foam and any combination thereof.

5. An implant according to claim 2, wherein said flexible container is comprised of a material selected from the group consisting of: mesh, suture, gel, porous, allograft, protein, hydrogel, collagen and spun fibers.

6. An implant according to claim 1, wherein said shape conformable substance is comprised of a material selected from the group consisting of: a surgical mesh, a woven material, a random fiber bundle, a flexible porous material, a liquid material, an allograft material, a protein material, a hydrogel material, a shape memory metal, and a self-expanding material.

7. An implant according to claim 1, wherein said shape conformable material is a surgical mesh comprising polyester and wherein said tissue insult mechanism is the surface texture of said polyester.

8. An implant according to claim 1, wherein said shape conformable material is a surgical mesh comprising polypropylene and wherein said tissue insult mechanism is the surface texture of said polypropylene.

9. An implant according to claim 1, wherein said shape conformable material is a woven material selected from the group consisting of a metallic material, a polymeric material, a bio-material, a bioresorbable material, and a non-bioresorbable material.

10. An implant according to claim 1, wherein said shape conformable material is a woven material and said tissue insult mechanism is a fibro-inductive agent sized to be released through openings in said woven material.

11. An implant according to claim 1, wherein said shape conformable material is a liquid material selected from a group consisting of collagen, tissue, thrombin, polymer, fibrin-based sealants and combinations thereof.

12. An implant according to claim 11, wherein said shape conformable material is collagen-thrombin sealant and said tissue insult mechanism is a collagen-reinforced liquid memostat.

13. An implant according to claim 11, wherein said shape conformable material is a thrombin based material and said tissue insult mechanism is platelet activation that initiates clot formation.

14. An implant according to claim 1, wherein said shape conformable material is an allograft material and the tissue insult mechanism is a matrix on said allograft material serving as a matrix for new tissue formation.

15. An implant according to claim 1, wherein said shape conformable material is a protein material and said tissue insult mechanism includes side chain elements located on said protein and said side chain elements regulating selective infiltration of said tissue.

16. An implant according to claim 1, wherein said shape conformable material is a hydrogel and said tissue insult mechanism includes a suspension of particles carried in said hydrogel.

17. An implant according to claim 1, wherein said shape conformable material is sized and shaped for positioning in approximately the lower ⅓ of the uterus.

18. An implant according to claim 1, wherein said shape conformable material is sized and shaped for positioning in approximately the lower ⅓ of the uterus and at least partially in the cervical canal.

19. An implant according to claim 1, wherein said shape conformable material is a stent.

20. An implant according to claim 19, wherein said tissue insult mechanism includes a predetermined expansion force loaded into said stent.

21. An implant according to claim 19, wherein said tissue insult mechanism includes a fibro-inductive coating on said stent.

22. An implant according to claim 19, wherein said tissue insult mechanism includes a tissue cooling mechanism disposed on said stent.

23. An implant according to claim 19, wherein said tissue insult mechanism includes a tissue heating mechanism disposed on said stent.

24. An implant according to claim 1, wherein said tissue insult mechanism includes a brittle coating disposed on said shape conformable substance

25. A method for treating undesired uterine bleeding comprising:
introducing a shape conformable substance into the uterus of a patient;
causing said shape conformable substance to insult the internal surface of the uterus; and,
allowing said shape conformable substance to remain in contact with said internal surface of the uterus until control of said undesired uterine bleeding is obtained.

26. A method as set forth in claim 25, wherein the introducing of a shape conformable substance includes introducing a flexible container containing a fibro inductive substance.

27. A method as set forth in claim 26, wherein said fibro-inductive agent is selected from the group consisting of: beads, spun fibers, bone particulate, cellulose, alginate, tissue, kitosan, particulate, composite, aggregate, foam and any combination thereof.

28. A method as set forth in claim 26, wherein said flexible container is comprised of a material selected from the group consisting of: mesh, suture, gel, porous, allograft, protein, hydrogel, collagen and spun fibers.

29. A method as set forth in claim 25, wherein said shape conformable substance is comprised of a material selected from the group consisting of: a surgical mesh, a woven material, a random fiber bundle, a flexible porous material, a liquid material, an allograft material, a protein material, a hydrogel material, a shape memory metal, and a self-expanding material.

30. A method according to claim 25, wherein the introducing of said shape conformable material includes introducing a surgical mesh comprising polyester and wherein causing of said insult includes contacting a surface texture of said polyester to contact uterine tissue.

31. A method according to claim 25, wherein the introducing of said shape conformable material includes introducing a surgical mesh comprising polypropylene and wherein causing said insult includes contacting a surface texture of said polypropylene to contact uterine tissue.

32. A method according to claim 25, wherein the introducing of said shape conformable material includes introducing a woven material selected from the group consisting of a metallic material, a polymeric material, a bio-material, a bioresorbable material, and a non-bioresorbable material.

33. A method according to claim 25, wherein the introducing of said shape conformable material includes introducing a woven material and wherein the causing of said insult includes releasing a fibro-inductive agent through openings in said woven material.

34. A method according to claim 25, wherein the introducing of said shape conformable material includes introducing a liquid material selected from a group consisting of collagen, tissue, thrombin, polymer, fibrin-based sealants and combinations thereof.

35. A method according to claim 25, wherein the introducing of said shape conformable material includes introducing is collagen-thrombin sealant and wherein the causing of said insult includes creating a collagen-reinforced liquid hemostat.

36. A method according to claim 25, wherein the introducing of said shape conformable material includes introducing a thrombin based material and wherein the causing of said insult includes inducing platelet activation that initiates clot formation in said uterus.

37. A method according to claim 25, wherein the introducing of said shape conformable material includes introducing an allograft material and wherein the causing of said insult includes using a matrix on said allograft material to serve as a matrix for new tissue formation in said uterus.

38. A method according to claim 25, wherein the introducing of said shape conformable material includes introducing a protein material and wherein the causing of said insult includes using side chain elements located on said protein to regulate selective infiltration of said tissue.

39. A method according to claim 25, wherein the introducing of said shape conformable material includes introducing a hydrogel and wherein the causing of said insult includes introducing a suspension of particles carried in said hydrogel into said uterus.

40. A method according to claim 25, wherein the introducing of said shape conformable material includes introducing said shape conformable material in approximately the lower ⅓ of the uterus.

41. A method according to claim 25, wherein the introducing of said shape conformable material includes introducing said shape conformable material in approximately the lower ⅓ of the uterus and at least partially in the cervical canal.

42. A method according to claim 25, wherein the introducing of said shape conformable material includes introducing a stent.

43. A method according to claim 42, wherein the causing of said insult includes expanding said stent into said uterine tissue.

44. A method according to claim 42, wherein the causing of said insult includes contacting a fibro-inductive coating on said stent with uterine tissue.

45. A method according to claim 42, wherein the causing of said insult includes cooling said tissue with said stent.

46. A method according to claim 42, wherein the causing of said insult includes heating said tissue with said stent.

47. A method according to claim 25, wherein the causing of said insult includes contacting a brittle coating on said shape conformable substance with said tissue.
